(19) United States
(12) Patent Application Publication
(10) Pub. No.: US 2022/0126128 A1
(43) Pub. Date: Apr. 28, 2022

(54) Lightweight Respiratory Mask

(71) Applicants: Christian Bayer, Penzberg (DE); Johannes Nickol, Stockheim (DE)

(72) Inventors: Christian Bayer, Penzberg (DE); Johannes Nickol, Stockheim (DE)

(73) Assignees: Christian Bayer, Penzberg (DE); Johannes Nickol, Stockheim (DE)

(21) Appl. No.: 17/512,318

(22) Filed: Oct. 27, 2021

(30) Foreign Application Priority Data
Oct. 28, 2020 (DE) 10 2020 006 605.4

(51) Int. Cl. A62B 18/02; A61M 16/06; A41D 13/11; A63B 71/10

(52) U.S. Cl. CPC A62B 18/025; A61M 16/0605; A41D 13/1107; A61M 2205/0233; A61M 2016/0661; A61M 2205/0238; A63B 71/10

(57) ABSTRACT

Disclosed is a particularly lightweight breathing mask for respiratory applications or as personal protective equipment, in which the rigid component is produced from thermoplastic films by means of high-pressure forming. The base material of the rigid component is available in a flat form and can thus be printed or coated in a variety of ways before processing; this allows properties to be introduced into the mask that would not be feasible in a conventional injection molding process. The use of film instead of injection-molded components enables a significant reduction in weight and thus an improvement in wearing comfort for the user. At the same time, the film allows a higher degree of flexibility of the rigid component during operation, enabling a better fit to the shape of the face under certain operating conditions and thus also improving wearing comfort

The present invention relates to a use in a respiratory therapy mask, a personal protective equipment mask, and/or a mask for sports and fitness applications and also for applications which reduce the spread of bacteria or viruses in the exhaled air of the wearer.

### TECHNOLOGICAL BACKGROUND OF THE INVENTION

Respiratory therapy masks usually consist of a mask cushion made of silicone or other elastic materials, which creates a seal to the patient's face. A therapeutically effective positive pressure can be applied via this seal. This form of therapy is known as CPAP therapy or non-invasive ventilation with or without leakage, among other names.

In order to obtain a closed cavity, the mask cushion is connected to a dome made of plastic. This dome also forms all of or at least part of the connection to the tubing system and the headgear that retains the mask on the patient's head. For both of these connections, a hard component is advantageous, for example, in order to create a rotatable joint to the tubing, to enable snap-on attachments, or to form retainers for the headgear.

Due to various, sometimes contradictory requirements for the mask dome, an additional frame is often used, which, for example, takes over the connection to the headgear, so in this case the dome is reduced to the functions of hose connection and creation of a cavity.

The hard component is usually manufactured by injection molding; this allows cost-effective production with a high degree of geometric freedom. However, there are also limitations associated with it, which can be improved by the present invention.

In applications in the field of personal protective equipment, a sealing effect must also be produced on the face of the wearer, so the invention can be used in this field in a similar manner as in the field of respiratory masks. However, the use of a filter medium instead of a respirator is the more likely application here. The attachment of a filter medium to a mask body of course also mandates the presence of a rigid body.

For applications in the sports and fitness sector, the comparatively low weight of the mask is of primary importance, but connections of filters, materials for increasing inhalation resistance, or exhalation valves are also common here, which can also be advantageously integrated into a rigid component.

In the area of reducing the spread of bacteria or viruses, low weight is particularly important, as is the ability to reprocess the material.

In the automotive sector, printed plastic films are often used, which are further processed in the injection molding process into decorative and/or functional elements in the vehicle interior as well as in the exterior. The printing or other coatings can have a purely decorative function, but can also use conductive inks to form electrical functional elements.

The films are often converted into a three-dimensional shape and trimmed before the injection molding process in order to achieve better processability. Deep-drawing/thermoforming processes or high-pressure forming (HPF), among others, are used here.

### DESCRIPTION OF THE INVENTION

A plastic film is shaped into a mask dome for respiratory masks by high-pressure forming (HPF) and then trimming.

The formed film can be combined with other elements, such as a mask cushion to seal on a wearer's face, a headgear, filters, tubes, exhalation valves, structures to rest on a wearer's face, or materials to influence flow characteristics in a controlled manner.

Some of the aforementioned components are themselves solids that can be attached, welded, or otherwise bonded to the rigid component formed from film. Others of said components are not inherently dimensionally stable, and thus require a rigid component for stabilization.

In the present invention, a film of thermoplastic, preferably high-temperature polycarbonate, is completely or partially overmolded with silicone rubber using a multicomponent injection molding process. The silicone overmolding can thereby form sealing lips, support structures, membranes or the like according to the prior art. The mask dome-mask cushion component can then be integrated into a mask system, for example, as would also be the case with a conventional injection-molded part.

The use of a formed plastic film has several advantages over the use of a plastic part which has been injection molded according to the prior art.

The main advantage is a weight saving: a formed film is in the range of 0.1 mm to 1.0 mm wall thickness, preferably in the range 0.4 mm to 0.6 mm. Compared to a conventional injection-molded part, which has a wall thickness of 1.0 mm to 2.5 mm, for example, a significant reduction in weight can thus be achieved. This makes the mask lighter and therefore more comfortable to wear for the user. The material savings also result in a reduction in costs.

Another advantage lies in the pliability or flexibility of the rigid component formed from foil. Due to the reduced thickness of the material, the film has greater flexibility or pliability when suitably shaped, this allows, for example, controlled deformation during operation. This reduces the tendency to slip on the wearer's face in some operating conditions, such as lateral resting on a pillow during the wearer's night sleep, thus improving the sealing effect.

By specifically designing the geometry of the rigid component formed from film, improved conformability to different face shapes can also be achieved by using the controlled elastic bending of the film to cover a larger percentage of the group of potential wearers, thus reducing the number of different manufacturing tools required. As a result, a reduction in manufacturing costs can be achieved.

Due to the significantly reduced weight and increased flexibility of the formed plastic film, a significant improvement in wearer comfort can be achieved.

Another advantage of producing a rigid component of a mask formed from film using the HPF process as opposed to conventional thermoforming or deep-drawing is the improved dimensional accuracy and better precision, since the film can be formed more precisely and at a lower temperature in the HPF process. Accurate and low-variation forming of the geometry of the rigid component is required for subsequent overmolding in a silicone injection mold. Furthermore, important geometric elements such as a connector for filter cartridges or ventilation hoses/ventilation accessories can be produced that meet the strict requirements for roundness, dimensional accuracy and conicity, which could not be repeatedly ensured in a thermoforming process. This enables the use of standardized accessories, for example in the field of respiratory medicine, and raises the formed sheet to a level of accuracy equivalent to an injection molded part.

Another advantage of the invention is the ability to apply a printing or other coating. The ability to print or coat the film prior to forming, and thus create a bond that will withstand the forming process and the downstream injection molding process, opens up a variety of possibilities that make the dome formed from film in the HPF process stand out from the prior art.

Printing designs on the finished part can be made far more complex, accurate, repeatable and aesthetically pleasing than a subsequent printing on an injection molded part using, for example, the pad printing process could possibly achieve. This opens up new aesthetic design opportunities. Due to the lower temperatures involved in using the HPF process compared to thermoforming, the printing or coating can be carried out more consistently and more gentle on the materials and inks used, as suggested by the state of the art in this field.

Special inks can be used which contain a fading effect, for example over time or after a number of cleaning cycles. Thus, a mask can be equipped with an optical wear indicator. The application of these inks prior to the forming process allows this advantageous element to be incorporated in a much more cost-effective manner than would be the case, for example, with the subsequent printing of an injection-molded part.

Because the film is flat before forming, conductive tracks can be printed, laminated, coated or otherwise applied, which would not be possible with a three-dimensionally shaped injection molded part. At the same time, the lower temperature of the HPF process compared to thermoforming prevents damage to the conductive track. The processing of a plastic film in the HPF process thus brings the possibility of integrating sensor technology or other electronic components into a mask system in a cost-effective manner, for example for temperature, humidity or pressure measurement.

Capacitive switches can be printed which, for example, allow a simple form of remote control of a connected device by the wearer of the mask, such that on-off functions, humidifier output or the like could be controlled from the mask so that the wearer does not have to reach over to the attached device first. This will improve the comfort of the entire application.

Conductive tracks applied to the mask body can support RFID applications, for example, so that breathing masks can be coded to specific device types. This provides additional security against incorrect operation.

The ability to apply a print or other coating to a flat film prior to the forming process allows a much more practical and cost-effective application of the various inks or coatings than would be possible with a subsequent application of the same functionality to a finished injection molded part. In addition, some of the above applications would most likely not be able to be efficiently applied to a three-dimensionally shaped injection molded part in the first place.

Overall, processing in the HPF process is advantageous over the thermoforming process because the forming can take place at significantly lower temperatures. As a result, pre-applied inks, coatings or conductive elements are subject to less thermal stress and are reproduced with greater accuracy.

### DESCRIPTION OF DRAWINGS

FIG. 1 shows an isometric view (11) of the formed rigid dome structure as seen from the outside, as it would be seen on a wearer's face, and another isometric view (12) as seen from inside the mask cushion cavity, the enclosure on the wearer's face. Several features are displayed here as examples, yet other shapes or embodiments are obviously also feasible. The rigid dome structure forms a part (111) of an enclosure on a wearer's face during use together with a subsequently overmoulded sealing portion, which is not shown here. Geometric features for receiving e.g. a wearer's nose (113) or chin (114) can be included. The rigid dome structure incorporates a planar flange (112) for forming part of a rotational interface to an air supply tubing. It also forms a flange area (115) along its outer perimeter for bonding to an elastic cushion portion during overmoulding. A conical structure (121) such as e.g. a standardized cone for connecting to respiratory tubing and accessories, is also formed as part of the rigid dome structure.

FIG. 2 shows a front view (21), a side view (22) and a cross-sectional view (23) of the formed rigid dome structure, again depicting the elements shown in FIG. 1, such as the planar flange (112) in (221), and the conical structure (121) in 231. FIG. 2 illustrates the very thin wall sections 232 achievable by forming a plastic film.

FIG. 3 shows two different embodiments of the rigid dome structure arrayed on a film sheet (33), the film sheet being characterized by an outer area (331) intended for retention in the forming tool, the inner area (332) enabling the deformation of the flat film sheet into the desired three-dimensional shape. One embodiment (31) shows a design more suitable for respiratory masks having a connection portion for an air supply tubing (312), as shown in FIG. 1 and FIG. 2, while the other embodiment is not intended to connect to an external air supply, such as might be the case in sports or personal protective equipment. In a manufacturing step subsequent to the high-pressure forming, both embodiments are trimmed at an external contour (311, 321), while the first embodiment (31) also is trimmed at an inner contour (312), to achieve the finished rigid dome structure which is then overmoulded with a sealing or face contact portion.

FIG. 4 shows a cross sectional view (41) through the formed film already shown in FIG. 3. A flat plastic material (411) is heated and formed into a three-dimensional shape by application of an elevated air pressure as known from the prior art. The film can be formed into a negative, or downward portion (412) of the forming tool, and the central areas of the forming tool can be raised (413) above the parting plane (414) so that it already forms a portion of the three-dimensional shape as the tool closes. Both effects combined allow for a maximized height of the formed rigid dome structure, while ensuring a uniform film stretch. In the present embodiment, the high-pressure forming tool is located on the lower side (415) of the film sheet, it is however obvious that the forming tool could also be located on the opposite side (416), so that the film sheet is formed face down. Such a face down forming may be advantageous in case conductive tracks or sensitive inks or coatings are pre-applied to the film, which cannot endure contact with the forming tool.

FIG. 5 shows another cross sectional view (51) of the formed rigid dome structure shown in the previous figures, depicting a second layer (511), e.g. a printing or coating layer, applied to the outside of the film prior to the high-pressure forming process. As the high-pressure forming process is dimensionally more accurate and more repeatable than vacuum forming or low-pressure forming, it is possible to position the second layer such that important areas of the formed rigid dome structure are not coated in the finished part, such as the flange intended for bonding during overmoulding (512) or the conical standardized connector portion (513). While FIG. 5 shows a coating applied to the outside of the formed rigid structure, it is obvious that printings or coatings may also be applied to an inside portion (514) of said structure, or to both the outside and the inside portions.

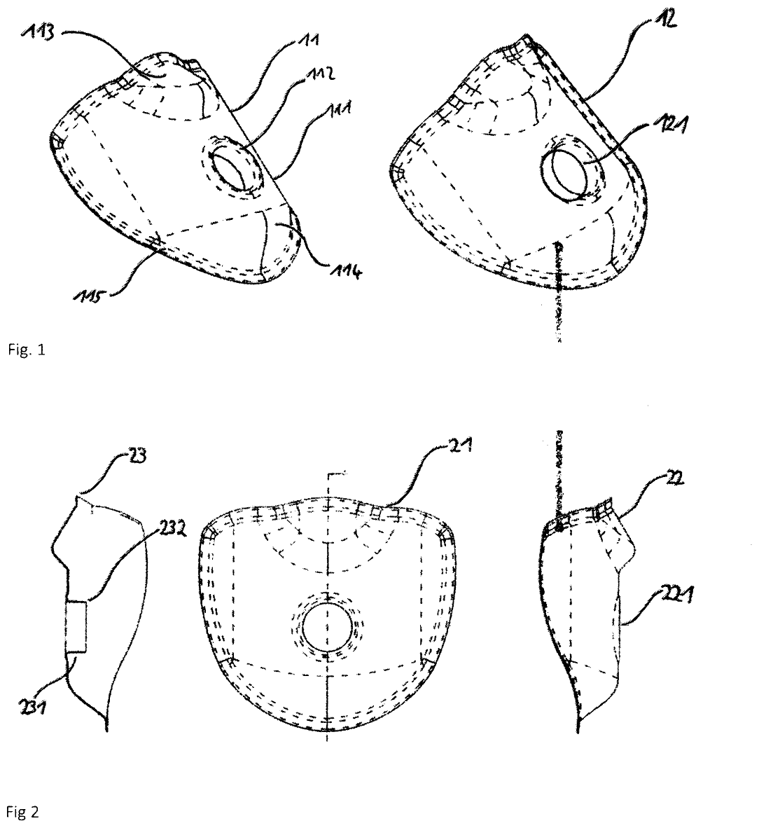

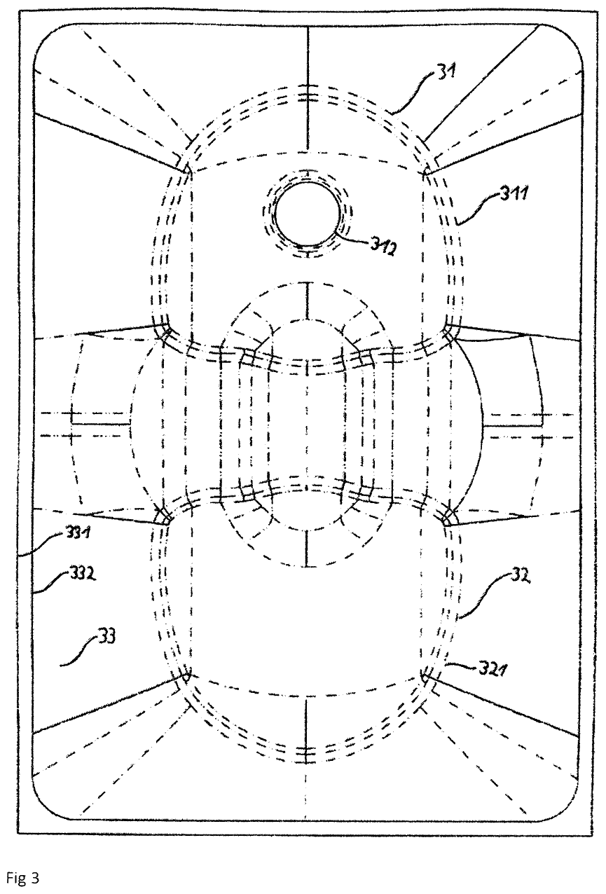

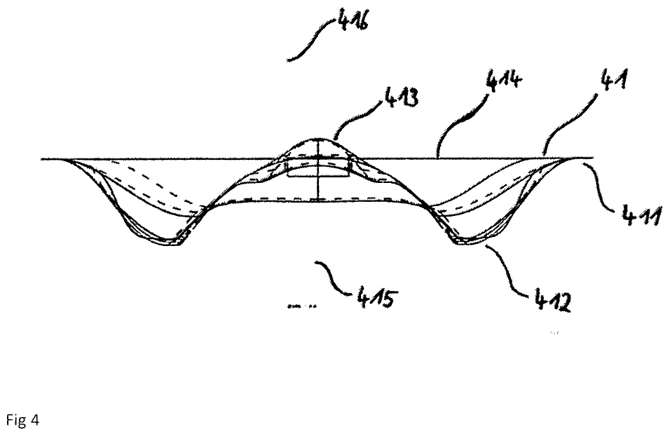

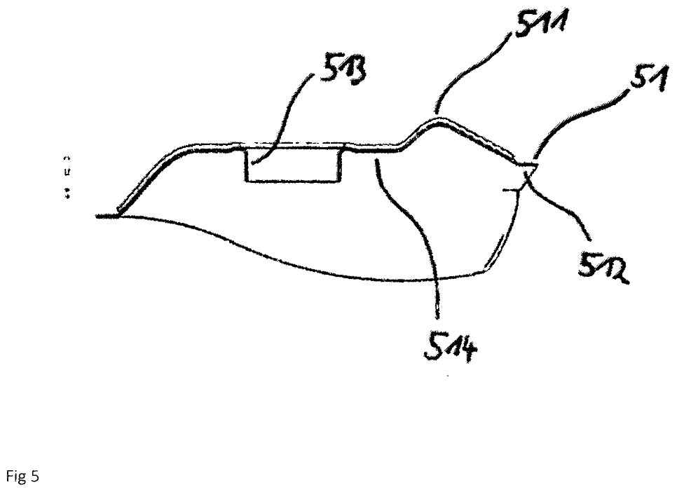

It is claimed:

1. a hollow dome made of plastic, preferably transparent plastic, for use in a respirator or respiratory therapy mask or in sports equipment or personal protective equipment, wherein the base material of the dome is present in the form of a plastic film which is formed into a three-dimensionally shaped form by means of high-pressure forming and is then trimmed to the required outer and optionally inner contours.

2. a hollow dome according to claim 1, which has a significantly lower weight, preferably 0.2 to 0.6 times the weight, more preferably 0.3 to 0.5 times the weight of a comparably shaped injection-molded dome, and thereby improves the comfort for the user.

3. a hollow dome according to claim 1, wherein the starting material, which comes in the form of a plastic film, has a thickness in the range from 0.1 mm to 1.0 mm, preferably from 0.3 mm to 0.7 mm, more preferably from 0.4 mm to 0.6 mm.

4. a hollow dome according to claims 1-3, wherein the starting material consists of a thermoplastic, preferably polycarbonate, further preferably high temperature polycarbonate.

5. a hollow dome according to claims 1-4, in which the rigid component retains a degree of flexibility after forming, so that a controlled elastic deformation can take place in operation which positively influences the wearing comfort on the face.

6. a degree of flexibility of a film formed into a hollow dome according to claim 5, which allows an elastic deformation between 10% and 50%, preferably between 20% and 40%.

7. a hollow dome according to claims 1-6, which is joined to a sealing lip of silicone or thermoplastic elastomer to achieve a sealing effect on the face of the support, and in which the sealing lip is either attached to the formed film, or is preferably injection molded to the formed film by a multicomponent injection molding process.

8. a hollow dome according to claims 1-6 as well as according to claim 7, wherein the starting material is coated prior to forming, preferably with one or more printing inks, which are maintained on the three-dimensional shape of the hollow dome after forming of the film.

9. a coating according to claim 8, which includes electrically conductive inks

10. a coating according to claim 9, in which electrical components are also applied to the starting material before forming.

11. an application of electrically conductive inks to the base material of a hollow dome according to claims 9-10, by means of which conductive tracks for capacitive switches are formed in the hollow dome of the mask which in turn can be used to control devices connected to the mask, for example respirators.

12. an application of electrically conductive inks to the base material of a hollow dome according to claims 9-11, in which the inks form contacts for subsequently applied sensor elements.

13. an application of electrically conductive inks to the base material of a hollow dome according to claims 9-12, in which the inks form conductive paths as antennas for short-range wireless communications.

14. a hollow dome according to claims 1-8, wherein printing inks are applied that wear, fade, abrade or intentionally delaminate over time.

15. a wear indicator according to claim 14, in which the wear of the printing ink occurs continuously through normal handling and cleaning of the hollow dome, for example through cleaning agents or UV light, and thus visually indicates the service life of the mask system.

16. a hollow dome according to claims 1-7, wherein the film is coated with a second material, such as wood, metal or cellular fibers, sufficiently thin to also be converted into a three-dimensional shape upon forming.
